(19) United States
(12) Patent Application Publication
(10) Pub. No.: US 2009/0182315 A1
(43) Pub. Date: Jul. 16, 2009

(54) LASER LIPOSUCTION SYSTEM AND METHOD

(75) Inventors: Angelika Zigan, Oberlakr (DE); Miklos Antal, Budapest (HU)

Correspondence Address: BOLESH J. SKUTNIK;CERAMOPTEC INDUSTRIES, INC., 515 SHAKER RD., EAST LONGMEADOW, MA, 01028, US

(73) Assignee: CeramOptec Industries Inc.

(21) Appl. No.: 12/329,308

(22) Filed: Dec. 5, 2008

Related U.S. Application Data
(60) Provisional application No. 61/005,767, filed on Dec. 7, 2007.

(51) Int. Cl. A61B 18/22

(52) U.S. Cl. 606/15

(57) ABSTRACT

A device and method for adipose tissue liquefaction and removal (liposuction) by means of laser energy is disclosed. The device comprises a medical laser system, an optical fiber, an innovative handpiece and a vacuum pumping system. The device is capable of liquefying and removing adipose tissue essentially simultaneously due to the innovative handpiece, which comprises an outer tube for adipose tissue extraction and an inner tube in which optical fiber is inserted and shielded. Furthermore, the optical fiber is isolated from external contamination with a transparent quartz tip or cap on the inner tube, which permits laser radiation output while avoiding liquid and tissue input. The transparent tip or cap can also be made of sapphire or fused synthetic silica. With this safer and improved process, enhanced liposuction treatments are possible with reduced bleeding, gentler and shorter treatment and quick patient recovery.

### DOMESTIC PRIORITY UNDER 35 USC 119(E)

This application claims the benefit of U.S. Provisional Application Ser. No. 61/005,767 filed Dec. 7, 2007, entitled “Laser Liposuction System and Method” by Angelika Zigan and Miklos Antal, which is incorporated by reference herein.

### BACKGROUND OF THE INVENTION

1. Field of the Invention

The present invention is related to minimally invasive devices and methods for treatment of biological tissue. More particularly, the invention relates to adipose tissue to liquefaction by means of laser irradiation and removal (liposuction).

2. Invention Disclosure Statement

Nowadays, the abundance of readily available foods and sedentary lifestyle allow people to gain excessive weight by an increase in adipose tissue fat cells. Sometimes, this situation is enhanced due to certain hereditary conditions.

Excessive fat deposits or lipodystrophies are produced by a disproportionate increase in the deeper section of the subcutaneous cellular tissues. The only effective way to treat lipodystrophies is to directly act on the genetically altered fat tissues and similar tissues in the treatment area.

Historically, different methods have been developed to treat this problem, i.e., direct liposuction, ultrasonic liposuction, vibrational liposuction, laser lipolysis, laser lipolysis and suction and simultaneous laser lipolysis and suction.

Direct liposuction consists of introduction into the adipose layers of probes roughly 5 mm in diameter through holes made in the skin of the patient undergoing treatment, for suction and removal of fat. This technique has a number of disadvantages, such as the in-homogeneity created in the zone of insertion of the probe, as well as excessive bleeding. Furthermore, both the cells of fat and the stroma are sucked out non-selectively. Several deaths have been reported because of the crudity of conventional liposuction.

Ultrasonic liposuction utilizes subcutaneous ultrasonic probes to rupture the membrane of the adipose cells, thus causing the escape of liquid which has to be sucked out subsequently by vacuum means, much like in direct liposuction. Liposuction by ultrasonic means also produces connective tissue damage, so bleedings might occur as well. Furthermore, the lack of homogeneity resulting from the treatment still remains as a disadvantage.

Vibration liposuction uses a vibrating handpiece with an extraction channel integrated. Tissue is extracted by vacuum means and can be removed faster in comparison with the before-mentioned methods. However, connective tissue is still damaged, thus bleeding and other long term problems can occur. Another disadvantage is that vibrations of the handpiece can stress a physician's wrist. Therefore, it becomes difficult for him to do precise work (e.g. suturation) after some time of treatment.

Laser lipolysis uses energy from a laser beam to liquefy the cells of the adipose layer. The liquefied fat is then carried away naturally by the lymphatic system or can be removed by compression of remaining tissue. In U.S. Pat. No. 5,954,710, Paolini et al. disclose a device for the removal of subcutaneous adipose layers which comprises a first laser source, an optical fiber for conveying the laser beam emitted by said first source and a hollow needle for guiding the fiber, the fiber ending in the vicinity of the end of the needle. Preferably, the laser source has a wavelength ranging from 750 nm to 2500 mm. Pulse energy level is about 100 mJ in 200 μs of pulse duration, during a treatment time of a few minutes. Liquefied tissue is sucked out or preferably left in place in order to be drained by lymphatic system and by action of phagocytes. According to Paolini et al., this method achieves a uniform outcome, no damage to stroma, and reduced bleeding due to laser cauterization of small blood vessels.

In U.S. Pat. Application No. 2006/0253112A1, Suarez et al. disclose a method and device for cosmetic surgery, especially fat reduction and collagen reformation, by means of a high power laser operating at about 980 nm. The cosmetic surgery method substantially reduces or removes localized lipodystrophies, and essentially reduces flaccidity (at least 50%, due to fibroelastic retraction) by localized laser heating of adipose tissue using an optical fiber inserted into a treatment area. High power laser energy is applied through an optical fiber for breakdown of fat cells walls releasing the cell fluid. The optical fiber may be held within a catheter-like device having a single lumen and may have a diffuser mounted on the tip to further apply heating to tissues surrounding the whole tip. A saline solution may also be inserted into the treatment site to aid in the heating of the fat cells and their eventual destruction as well as their removal. The pool of cell fluid in the area of treatment is removed by a combination of techniques including: body removal by absorption and drainage from the entry sites (minimizing trauma), direct force application by means of elastic bandages and external suction applied to the entry sites. According to Suarez et al., treatment with the 980 nm laser was efficient and more suitable than traditional liposuction on up to 80% of the patients.

In U.S. Pat. Application No. 2006/0224148, Cho et al. disclose a device and related method for the removal of subcutaneous adipose layers comprising a laser source; an optical fiber for conveying a laser beam emitted by the laser source; and a hollow cannula for guiding the fiber to the subcutaneous treatment area. The cannula has a curved portion at its distal end, where the curved portion can be shaped to roughly conform to the contour of the patient's body structure. In this way, laser energy from the fiber, applied to the adipose layers, is generally directed away from the lower dermis of the patient, minimizing the risk of non-reversible damage to the dermis, including skin necrosis. A radiation detector or a temperature sensitive material can be applied to the surface of the skin above the treatment area to warn of harmful dermal temperatures, triggering a cooling mechanism.

As tissue removal is restricted to the lymphatic system and compression of remaining tissue, only a low volume of tissue can be extracted effectively. Furthermore, the removal of liquefied adipose tissue via the lymphatic system can be insufficient and at times very dangerous.

The technique of liposuction after laser lipolysis, utilizes a laser source to liquefy adipose tissue and then removes this tissue by means of a vacuum source. This method enhances the amount of liquefied tissue removal in comparison to laser lipolysis alone. However, as the removal of tissue is done after lipolysis, an ultrasound post-treatment is often necessary for the extraction of remaining tissue, increasing treatment time and cost and adding complexity to the process.

Another approach for performing liposuction is the simultaneous lipolysis and tissue extraction technique, which utilizes a laser source to liquefy adipose tissue and an extraction means for tissue removal in a substantially simultaneous way. In U.S. Pat. No. 6,464,694, Massengill discloses a liposuction cannula having a source of aqueous solution, a laser source, and a suction source. Aqueous solution is released into an active area within the cannula, and laser energy is directed onto the aqueous solution within the active area to energize the water molecules. The energized water molecules escape from the active area into the surrounding fatty tissue to break down the fatty tissue and release liquid fatty material, which is removed by aspiration via the cannula. As can be seen, the device used in this invention does not apply laser energy in a direct way, so the amount of energy delivered can be difficult to quantify and can lead to indiscriminate tissue damage.

In U.S. Pat. No. 6,918,903, Bass discloses a device and method which allow simultaneous application of suction or vacuum for evacuation of fat with application of electrical bipolar energy to the fat inside an opening in a cannula. A pair of electrodes is situated within the cavity of the cannula just under the surface of cannula tip opening(s) or as part of the walls of such openings. The electrodes are spaced to allow coagulation of fat entering the cannula. Irrigation may be applied in a continuous or discontinuous or intermittent stream within the cannula to cool the tip and facilitate removal of suctioned tissue and prevent buildup of debris on electrodes.

In both afore-mentioned inventions, energy source is in direct contact with the removed tissue, thus contamination with its associated risks might occur. An unprotected energy source could be damaged, contaminated (changing specific characteristics of emitting surface) or broken. In case of breakage, the removal of fragments could be a very difficult task.

Due to the disadvantages and deficiencies of current liposuction techniques, a need exists for a device that provides a fast and safe alternative to address their shortcomings.

Some of the before-mentioned techniques limitations and problems can be overcome by a simultaneous and continuous lipolysis and liquefied tissue extraction technique, which utilizes a laser source to liquefy adipose tissue and an extraction means for tissue removal in a simultaneous way. This technique, as explained further below, presents some important advantages comparing to previous methods, i.e., shorter treatment time, larger volume of removed tissue, avoidance of possible adipose embolus, and less physician and patient stress.

### OBJECTIVES AND BRIEF SUMMARY OF THE INVENTION

It is an objective of the present invention to provide a device to allow for performance of a laser liposuction technique in which lipolysis and tissue removal are essentially simultaneous.

It is another objective of the present invention to provide a device in which the optical fiber is protected from damage, contamination and breakage.

It is still another objective of the present invention to provide a system for effective laser liposuction treatment which minimizes bleeding, various risks, treatment crudity and time, and patient recovery time.

It is yet another objective of the present invention to provide a method for effectively performing a laser liposuction technique in which lipolysis and tissue removal are substantially simultaneous, with improved safety for patients and device.

Briefly stated, a device and method for adipose tissue liquefaction and removal (liposuction) by means of laser energy is disclosed. The device comprises a medical laser system, an optical fiber, an innovative handpiece and a vacuum pumping system. The device is capable of liquefying and removing adipose tissue essentially simultaneously due to the innovative handpiece, which comprises an outer tube for adipose tissue extraction and an inner tube in which optical fiber is inserted and shielded. Furthermore, the optical fiber is isolated from external contamination with a transparent quartz tip or cap on the inner tube, which permits laser radiation output while avoiding liquid and tissue input. The transparent tip or cap can also be made of sapphire or fused synthetic silica. With this safer and improved process, enhanced liposuction treatments are possible with reduced bleeding, gentler and shorter treatment and quick patient recovery.

The above and other objects, features and advantages of the present invention will become apparent from the following description read in conjunction with the accompanying drawings, (in which like reference numbers in different drawings designate the same elements).

### BRIEF DESCRIPTION OF FIGURES

FIG. 1 depicts an embodiment of a laser liposuction system including an irradiation system, a low-pressure/vacuum system and a handpiece.

FIGS. 2a, 2b and 2c show a breakout of a preferred embodiment of a handpiece.

FIG. 3a shows an expanded view of the distal end of the handpiece embodiment of FIG. 2.

FIG. 3b depicts a view of a longitudinal section of part of the handpiece distal end.

FIG. 4 shows a representative flow diagram of a method embodiment of the present invention.

FIGS. 5a, 5b and 5c depict a preferred embodiment in which a side-emitting fiber is used.

FIGS. 6a, 6b and 6c show another preferred embodiment where the optical fiber tip is drop shaped.

FIGS. 7a, 7b and 7c depict a preferred embodiment in which inner tube is bent at its distal end.

FIG. 8 shows a schematic drawing of a detachable handpiece.

### DETAILED DESCRIPTION OF PREFERRED EMBODIMENTS

The present invention discloses a device and method which allows for a safer and more effective liposuction treatment, by means of laser lipolysis and essentially simultaneous adipose tissue removal.

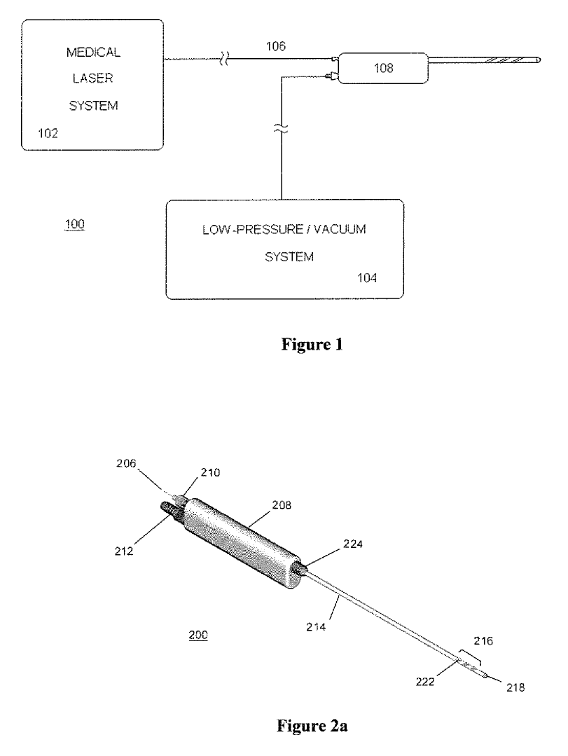

FIG. 1 shows laser liposuction system 100 comprising medical laser source 102, optical fiber 106, innovative handpiece 108 and vacuum pumping system 104. Both laser source 102 and vacuum pumping system 108 are connected to handpiece 108 in their respective tubes, for performing the liposuction treatment.

In a preferred embodiment, the medical laser source is a 980 nm/50 W laser. Laser radiation power is typically about 13 W to about 30 W, in a continuous mode. Pulsed laser emission is not used or required during this treatment. The optical fiber used in this preferred embodiment is a 600 μm fiber with a bare fiber tip.

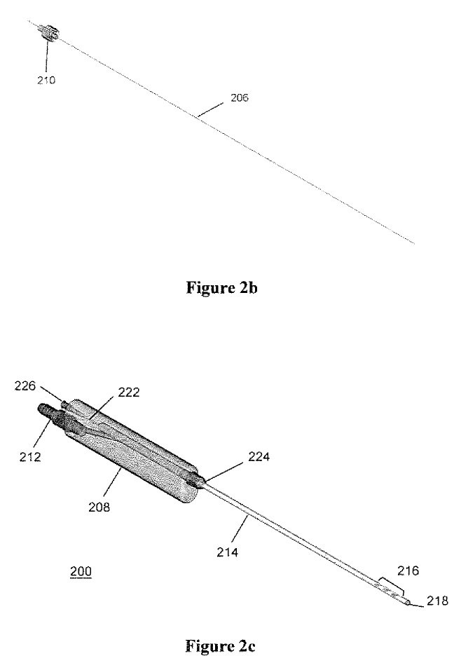

One preferred embodiment of a handpiece assembly is shown in FIGS. 2a, 2b and 2c. Handpiece 200 comprises two independent and isolated concentric metal tubes. Laser fiber 206 is inserted into inner metal tube 222 securing the proximal end of the optical fiber to the proximal end of the handpiece with for example a Luer lock 210. The distal end of the optical fiber is aligned with the handpiece distal end 218, to deliver laser energy. Liquefied adipose tissue removal is done through outer metal tube 214, with liquefied adipose tissue draining through its slits/holes 216 located near the distal end of the handpiece. By means of a vacuum source connected to outer metal tube 214 through connector 212, adipose tissue is removed from the handpiece.

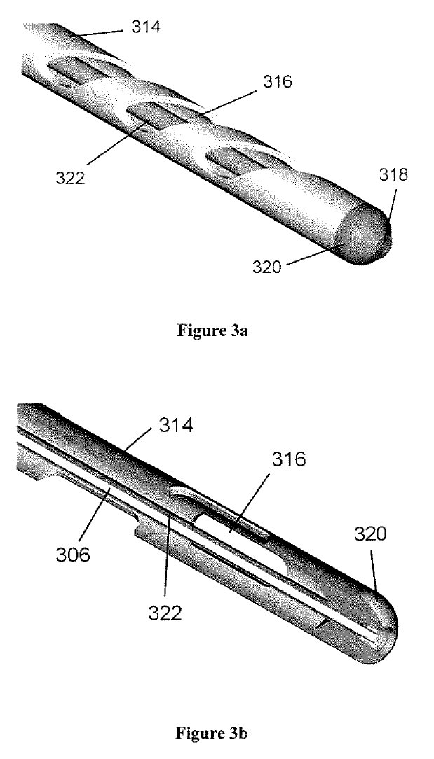

An expanded and detailed view of a preferred embodiment of the handpiece distal end is depicted in FIGS. 3a and 3b. Outer metal tube 314 having slits/holes 316 through which liquefied adipose tissue can drain from the patient under treatment to the vacuum source. As can be seen from the longitudinal section view, inner tube 322 and outer tube 314 are completely independent, so optical fiber 306 inserted into inner tube 322, is protected from tissue contamination, carbonization, damage and breakage. Once inserted into inner tube 322, optical fiber tip reaches handpiece tip 320. Handpiece tip 320 includes special quartz window 318 through which laser radiation is emitted.

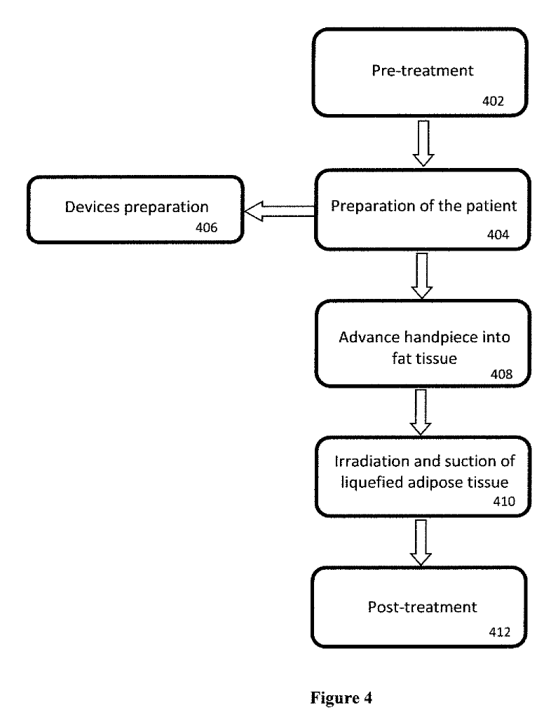

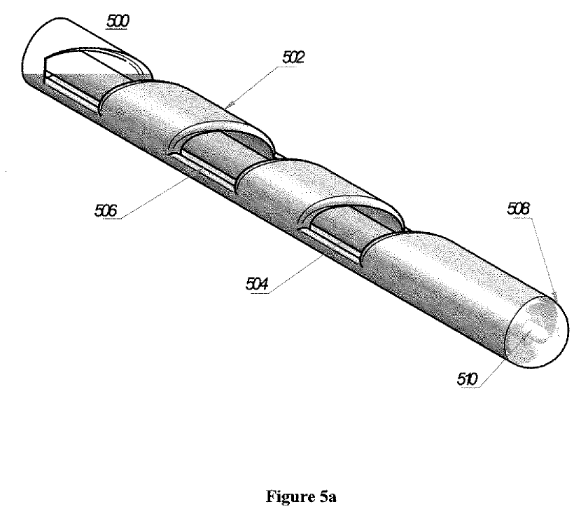

FIG. 4 shows an example of the method used in the present invention. During pre-treatment 402, patient must wear compressing tights for at least five days. The day of the treatment, devices are prepared (406) and patient gets general anesthesia previous to the laser procedure itself. After general anesthesia takes effect, the patient's skin is marked with a special marker in the area to be treated. Both anesthesia and skin marking are included in patient preparation 404. Accordingly, a small incision is done in the marked area of the skin and the handpiece is advanced into the fat tissue (408). Thereafter, laser is activated and vacuum pumping system is turned on. By subsequently moving handpiece back and forward continuously the adipose tissue is liquefied and removed (410). A certain part of the laser radiation heats up the distal end of the handpiece (where the quartz window is mounted). Due to the increased temperature of the distal end, the handpiece can be moved more easily backwards and forwards in the adipose tissue. After the treatment, it is strongly recommended that the patient wears compression tights for at least five days (412).

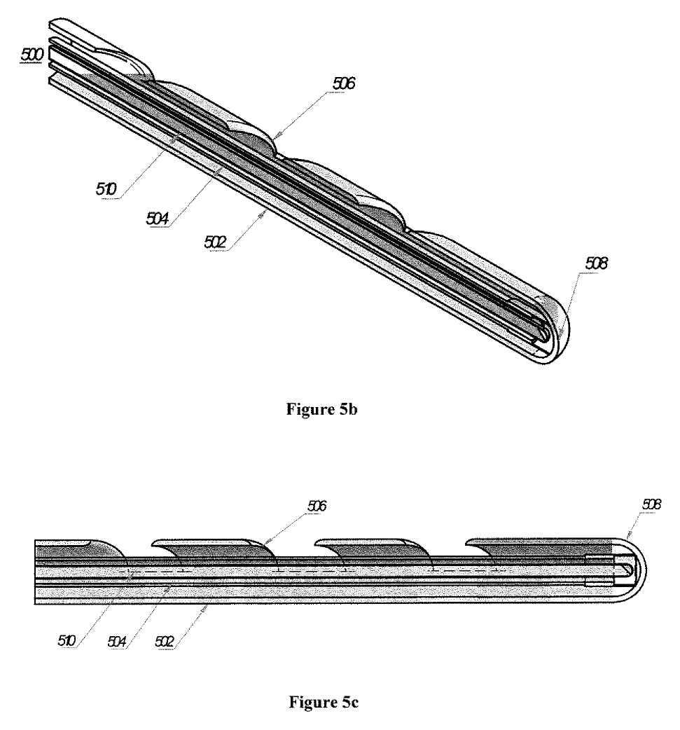

An expanded and detailed view of another preferred embodiment of the handpiece distal end is depicted in FIGS. 5a, 5b and 5c. Outer metal tube 502 comprises slits/holes 506 through which liquefied adipose tissue can drain from the patient under treatment to the vacuum source. In this embodiment, optical fiber 510, preferably a side-emitting fiber, is introduced into inner tube 504, which is completely independent from outer tube 502. Once inserted into inner tube 504, optical fiber tip reaches handpiece tip 508. Handpiece tip 508 is totally made of quartz, allowing radiation to be emitted in any direction in a plane perpendicular to its main axis, by rotating side fiber 510 inside inner tube 504 with no handpiece rotation needed. As a consequence, handpiece maneuverability is essentially improved and surgeon is able to gain better control on the treatment and to perform real shaping/sculpturing of the treatment area.

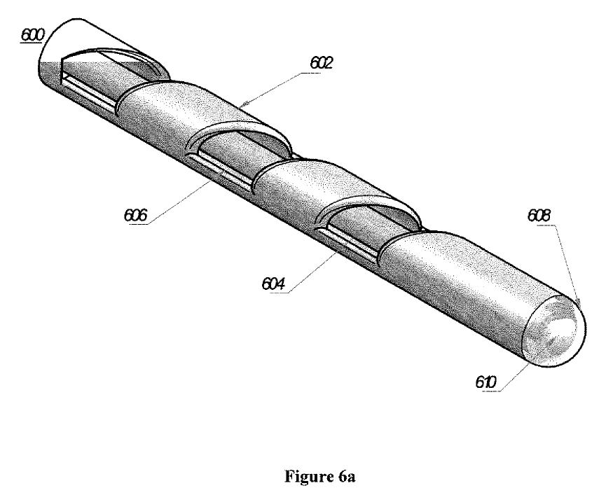

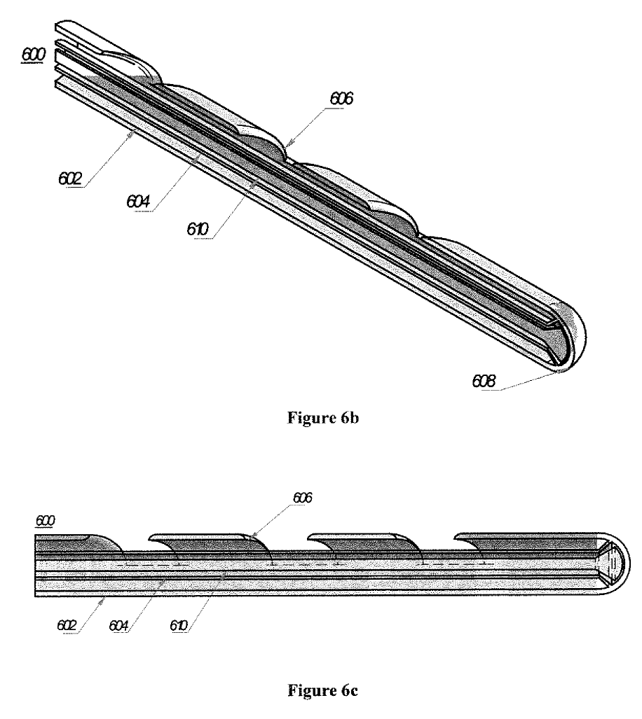

FIGS. 6a, 6b and 6c depict another preferred embodiment in which optical fiber 610 tip is drop shaped, thus emitting radiation in a diffused manner. This may be useful when surgeon needs to irradiate a larger less-specific treatment area, for instance, in patients with large volumes of adipose tissue. In this preferred embodiment, the handpiece-fiber set can be disposable.

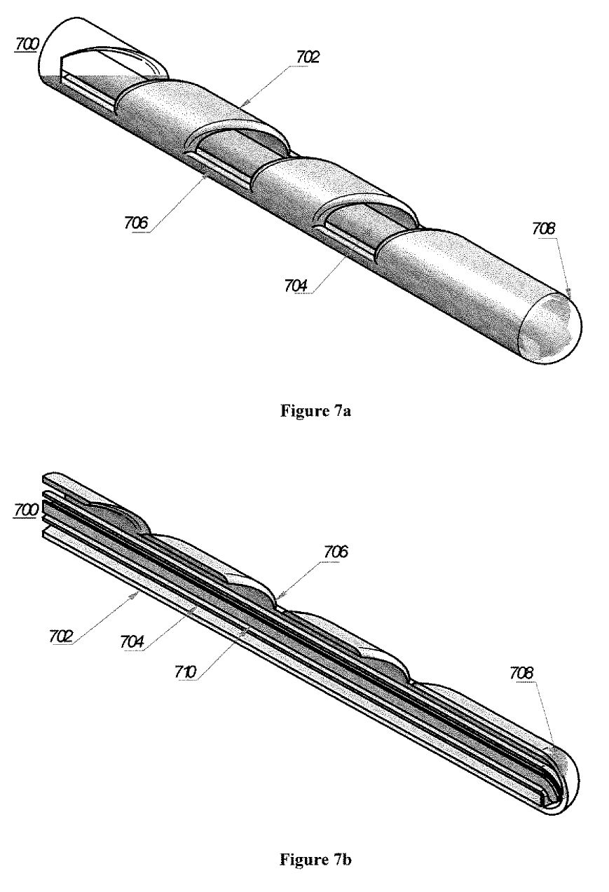

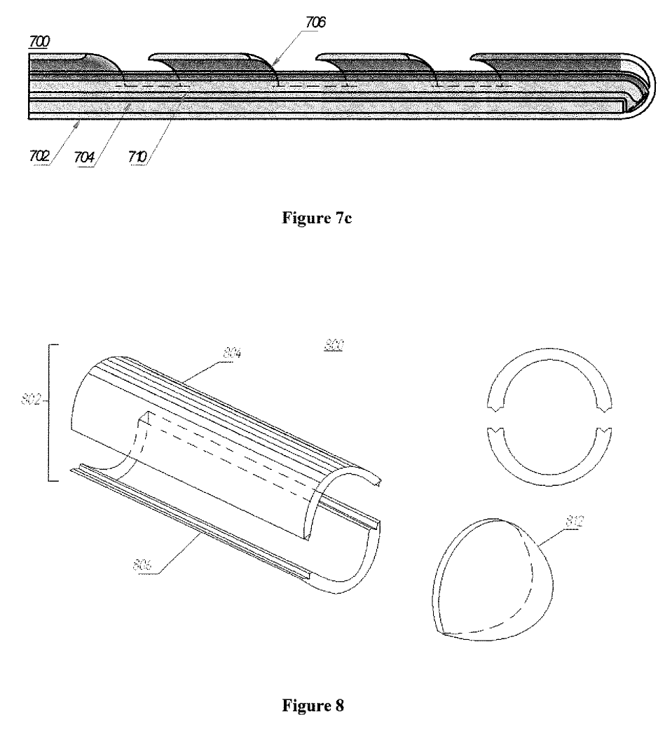

FIGS. 7a, 7b and 7c show another preferred embodiment in which inner tube 704 is bent at its distal end inside outer tube 702, ending in a quartz window facing towards one side of handpiece tip 708. This configuration causes laser radiation to be emitted perpendicularly to handpiece's main axis.

In another preferred embodiment, the quartz handpiece cap can be replaced by a similar material, such as sapphire, which is more durable, or fused synthetic silica.

In FIG. 8, a preferred embodiment can be seen showing a detachable handpiece. This figure depicts handpiece 800, in which outer tube 802 is split longitudinally, comprising upper half 804 and lower half 806. Both halves are held together by end cap 812. The end cap 812 is clipped to the proximal end of the outer tube 802. The detachable feature of this handpiece provides an important advantage, allowing for easier and more effective cleaning and sterilization.

The liposuction system disclosed in the present invention has numerous advantageous features. As the adipose tissue is liquefied and not vaporized or cracked, connective tissue and collagen fibers are not damaged, so bleeding is minimized greatly as its damage to the underlying structure.

Due to the simultaneous action of laser lipolysis and tissue extraction, this method is at least twice as fast as the vibration or other liposuction method and no vibration stress occurs in physician wrist. Furthermore, results are instantaneously observed, so surgeon is able to gain control on the treatment and to perform real shaping/sculpturing of the treatment area.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A laser liposuction system comprising:
a laser source;
an optical fiber having a proximal end and a distal end;
a combination handpiece having a proximal end and a distal end; and
a vacuum pumping system.

2. The laser liposuction system according to claim 1, wherein said laser source and said vacuum pumping system are separately connected to said combination handpiece

3. The laser liposuction system according to claim 1, wherein said laser source is selected from a group consisting of 980 nm laser or a 1470 nm laser.

4. The laser liposuction system according to claim 3, wherein said 980 nm laser source emits continuous radiation power from about 13 W to about 30 W.

5. The laser liposuction system according to claim 1, wherein said optical fiber is a 600 μm fiber with a bare tip.

6. The laser liposuction system according to claim 1, wherein said optical fiber is a side-emitting fiber.

7. The laser liposuction system according to claim 1, wherein said optical fiber has a drop shaped tip.

8. The laser liposuction system according to claim 1, wherein said combination handpiece comprises at least two concentric tubes an inner tube and an outer tube, which travel essentially the entire length of said handpiece, and terminate at said distal end of said handpiece.

9. The laser liposuction system according to claim 8, wherein the distal end of said inner tube is bent.

10. The laser liposuction system according to claim 8, wherein said optical fiber is positioned within said inner tube of said handpiece.

11. The laser liposuction system according to claim 1, wherein said proximal end of said optical fiber is fixed to said proximal end of said handpiece.

12. The laser liposuction system according to claim 1, wherein said distal end of said optical fiber is positioned near said distal end of said handpiece.

13. The laser liposuction system according to claim 8, wherein said inner tube of said handpiece has a transparent cap where said inner tube terminates at said distal end of said handpiece.

14. The laser liposuction system according to claim 13, wherein said transparent cap is made from the group consisting of quartz, sapphire or fused synthetic silica, allowing laser radiation output while avoiding liquid and tissue input.

15. The laser liposuction system according to claim 13, wherein said optical fiber, optically connected to said laser source, transmits energy from said laser source to and through said transparent cap and into a treatment area, where excessive adipose tissue, that needs to be removed, is present.

16. The laser liposuction system according to claim 13, wherein said distal end of said bent inner tube allows said transparent cap to face towards one side of said distal end of said handpiece, thereby emitting radiation perpendicular to the main axis of said handpiece.

17. The laser liposuction system according to claim 8, wherein said outer tube of said handpiece has at least one opening to accept liquefied adipose tissue, which is then evacuated by said vacuum pumping system.

18. The laser liposuction system according to claim 17, wherein near said distal end of said handpiece said outer tube has at least one opening to accept liquefied adipose tissue, which is then evacuated by said vacuum pumping system.

19. The laser liposuction system according to claim 1, wherein said distal end of said handpiece has a transparent cap.

20. The laser liposuction system according to claim 19, wherein said transparent cap is made of a material selected from the group consisting of quartz, sapphire and synthetic fused silica, and through which laser radiation is emitted.

21. The laser liposuction system according to claim 20, wherein said quartz transparent cap allows laser radiation to be emitted in any direction in a plane perpendicular to said transparent cap's main axis, by rotating said side-emitting fiber inside said inner tube of said handpiece, while simultaneously not rotating said handpiece.

22. The laser liposuction system according to claim 1, wherein said handpiece is detachable.

23. The laser liposuction system according to claim 22, wherein said outer tube of said handpiece is split longitudinally, comprising an upper half and a lower half,

23. The laser liposuction system according to claim 23, wherein said upper half and said lower half of said handpiece are held together by an end cap.

24. A method of improved laser liposuction, where preferably area to be treated is preferably marked on a patient's skin, comprising the following steps:
(i) introduce a handpiece according to claim 1 into a treatment area through a small incision within said marked area of said patient's skin;
(ii) activate said laser source of claim 1 and set power level;
(iii) irradiate said treatment area, essentially continuously, using a back and forth motion, while also evacuating liquefied adipose tissue with said vacuum pumping system of claim 1; and
(iv) continue irradiation and evacuation of adipose tissue until said treatment area is substantially rid of excess adipose tissue.

25. The method of laser liposuction according to claim 24 comprising the further steps of: prior to step (i) patient wears compression tights for at least 5 days prior to treatment, and a general anesthetic is administered prior to treatment; and after step (iv) patient wears compression tights for at least 5 days post treatment.
